(19) United States
(12) Patent Application Publication
(10) Pub. No.: US 2016/0159718 A1
(43) Pub. Date: Jun. 9, 2016

(54) MONOPHOSPHITES COMPRISING AN ANTHROL

(71) Applicants: Katrin Marie Dyballa, Recklinghausen (DE); Robert Franke, Marl (DE); Frank Geilen, Haltern am See (DE); Dirk Fridag, Haltern am See (DE); Dieter Hess, Marl (DE); Detlef Selent, Rostock (DE); Armin Boerner, Rostock (DE)

(72) Inventors: Katrin Marie Dyballa, Recklinghausen (DE); Robert Franke, Marl (DE); Frank Geilen, Haltern am See (DE); Dirk Fridag, Haltern am See (DE); Dieter Hess, Marl (DE); Detlef Selent, Rostock (DE); Armin Boerner, Rostock (DE)

(73) Assignee: Evonik Degussa GmbH, Essen (DE)

(21) Appl. No.: 14/953,210

(22) Filed: Nov. 27, 2015

(30) Foreign Application Priority Data
Dec. 4, 2014 (EP) 14196192.0

(51) Int. Cl. C07C 45/50; B01J 31/18; C07B 41/06; C07F 9/6574; C07F 15/00

(52) U.S. Cl. CPC C07C 45/50; C07F 9/65744; C07F 15/0073; C07B 41/06; B01J 31/185; B01J 2231/321; B01J 2531/822

(57) ABSTRACT

Monophosphites comprising an anthrol are useful for catalyzing hydroformylation of an olefin to an aldehyde.

### BACKGROUND OF THE INVENTION

1. Field of the Invention

The invention relates to monophosphites comprising an anthrol. In addition, the use thereof as ligands in hydroformylation.

2. Discussion of the Background

The reactions between olefin compounds, carbon monoxide and hydrogen in the presence of a catalyst to give the aldehydes comprising one additional carbon atom are known as hydroformylation or oxo synthesis. In these reactions, compounds of the transition metals of group VIII of the periodic table of the elements are frequently employed as catalysts. Known ligands are, for example, compounds from the classes of the phosphines, phosphites and phosphonites, each with trivalent phosphorus P(III). A good overview of the state of the art in the field of olefin hydroformylation may be found in B. CORNILS, W. A. HERRMANN, “Applied Homogeneous Catalysis with Organometallic Compounds”, vol. 1 & 2, VCH, Weinheim, N.Y., 1996 or R. Franke, D. Selent, A. Borner, “Applied Hydroformylation”, Chem. Rev., 2012, DOI:10.1021/cr3001803.

Every catalytically active composition has its specific benefits. According to the feedstock and target product, therefore, different catalytically active compositions are used.

EP 0 155 508 A1 discloses the use of bisarylene-substituted monophosphites in the rhodium-catalysed hydroformylation of sterically hindered olefins, e.g. isobutene. However, rhodium concentrations used here are sometimes very high (one being 250 ppm), which is unacceptable for an industrial scale process in view of the current cost of rhodium and has to be improved. A compound is shown on page 41 of the above, in which three phenyl radicals are each attached to one another via a C—C bridge, in this case taking the form of a 2,6-biphenylphenol unit.

### SUMMARY OF THE INVENTION

It was an object of the invention to provide monophosphites having advantageous properties compared to the known monophosphites in the hydroformylation reaction. In particular, the object consists of providing novel ligands having, in addition to the biphenol unit, further aromatic systems, which lead to an improved yield compared to the use of structurally related monophosphites. The improved yield should be achieved for at least one olefin.

A metal concentration (for example rhodium) of less than 100 ppm is also desirable.

The present invention provides a compound having one of the structures I or II:

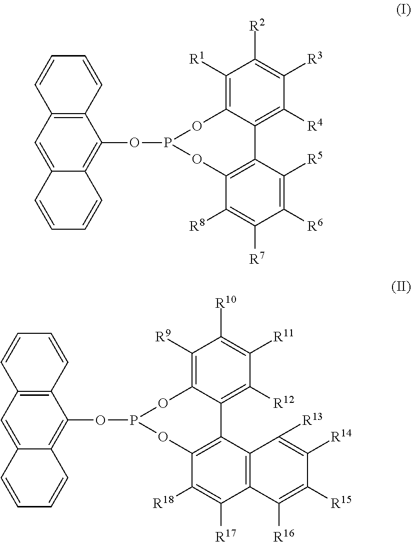

wherein

R¹, R², R³, R⁴, R⁵, R⁶, R⁷, R⁸ are each independently selected from the group consisting of:

—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —CN, —NH₂, and —N[(C₁-C₁₂)-alkyl]₂;

R⁹, R¹⁰, R¹¹, R¹², R¹³, R¹⁴, R¹⁵, R¹⁶, R¹⁷, R¹⁸ are each independently selected from the group consisting of:

—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —NH₂, and —N[C₁-C₁₂)-alkyl]₂;

wherein the alkyl and aryl groups may be substituted.

In one embodiment, the invention provides a complex, comprising:

a compound as above; and

a metal atom selected from the group consisting of: Rh, Ru, Co, and Ir.

The invention also relates to a catalyst for catalyzing a hydroformylation reaction, comprising: the compound as above.

The invention further relates to a process for hydroformylation of an olefin, comprising:

a) initially charging an olefin into a reactor;

b) adding
i) a complex as above;
or
ii) a compound as above and a substance having a metal atom selected from the group consisting of: Rh, Ru, Co, and Ir;

c) feeding into said reactor H₂ and CO, to obtain a reaction mixture;

d) heating the reaction mixture, to obtain conversion of the olefin to an aldehyde.

### DETAILED DESCRIPTION OF THE INVENTION

The object is achieved by a compound according to the present invention which provides a compound having one of the general structures (I) or (II):

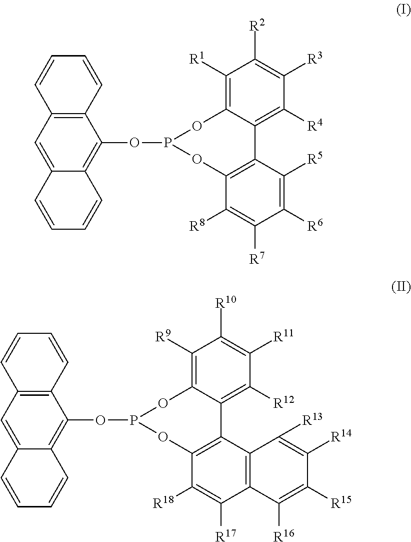

where

R¹, R², R³, R⁴, R⁵, R⁶, R⁷, R⁸ are selected from:

—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —CN, —NH₂, and —N[(C₁-C₁₂)-alkyl]₂;

R⁹, R¹⁰, R¹¹, R¹², R¹³, R¹⁴, R¹⁵, R¹⁶, R¹⁷, R¹⁸ are selected from:

—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —NH₂, and —N[C₁-C₁₂)-alkyl]₂;

wherein the alkyl and aryl groups may be substituted.

Any ranges herein below include all values and subvalues between the lowest and highest limits of the range.

(C₁-C₁₂)-Alkyl and O—(C₁-C₁₂)-alkyl may each be unsubstituted or substituted by one or more identical or different radicals selected from (C₃-C₁₂)-cycloalkyl, (C₃-C₁₂)-heterocycloalkyl, (C₆-C₂₀)-aryl, fluorine, chlorine, cyano, formyl, acyl and alkoxycarbonyl.

(C₆-C₂₀)-Aryl and —(C₆-C₂₀)-aryl-(C₆-C₂₀)-aryl- may each be unsubstituted or substituted by one or more identical or different radicals selected from:

—H, —(C₁-C₁₂)-alkyl, —O—(C₁-C₁₂)-alkyl, —O—(C₆-C₂₀)-aryl, —(C₆-C₂₀)-aryl, -halogen (such as Cl, F, Br, I), —COO—(C₁-C₁₂)-alkyl, —CONH—(C₁-C₁₂)-alkyl, —(C₆-C₂₀)-aryl-CON[(C₁-C₁₂)-alkyl]₂, —CO—(C₁-C₁₂)-alkyl, —CO—(C₆-C₂₀)-aryl, —COOH, —OH, —SO₃H, —SO₃Na, —NO₂, —CN, —NH₂, —N[(C₁-C₁₂)-alkyl]₂.

In the context of the invention, the expression “—(C₁-C₁₂)-alkyl” encompasses straight-chain and branched alkyl groups. Preferably, these groups are unsubstituted straight-chain or branched —(C₁-C₈)-alkyl groups and most preferably —(C₁-C₆)-alkyl groups. Examples of —(C₁-C₁₂)-alkyl groups are especially methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1,1,2-trimethylpropyl, 1,2,2-trimethylpropyl, 1-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The elucidations relating to the expression “—(C₁-C₁₂)-alkyl” also apply to the alkyl groups in —O—(C₁-C₁₂)-alkyl, i.e. in —(C₁-C₁₂)-alkoxy. Preferably, these groups are unsubstituted straight-chain or branched —(C₁-C₆)-alkoxy groups.

Substituted —(C₁-C₁₂)-alkyl groups and substituted —(C₁-C₁₂)-alkoxy groups may have one or more substituents, depending on their chain length. The substituents are preferably each independently selected from —(C₃-C₁₂)-cycloalkyl, —(C₃-C₁₂)-heterocycloalkyl, —(C₆-C₂₀)-aryl, fluorine, chlorine, cyano, formyl, acyl and alkoxycarbonyl.

The expression “—(C₃-C₁₂)-cycloalkyl”, in the context of the present invention, encompasses mono-, bi- or tricyclic hydrocarbyl radicals having 3 to 12, especially 5 to 12, carbon atoms. These include cyclopropyl-, cyclobutyl-, cyclopentyl-, cyclohexyl-, cycloheptyl-, cyclooctyl-, cyclododecyl-, cyclopentadecyl-, norbonyl- and adamantyl.

One example of a substituted cycloalkyl would be menthyl.

The expression “—(C₃-C₁₂)-heterocycloalkyl groups”, in the context of the present invention, encompasses nonaromatic saturated or partly unsaturated cycloaliphatic groups having 3 to 12, especially 5 to 12, carbon atoms. The —(C₃-C₁₂)-heterocycloalkyl groups have preferably 3 to 8, more preferably 5 or 6, ring atoms. In the heterocycloalkyl groups, as opposed to the cycloalkyl groups, 1, 2, 3 or 4, the ring carbon atoms are replaced by heteroatoms or heteroatom-containing groups. The heteroatoms or the heteroatom-containing groups are preferably selected from —O—, —S—, —N—, —N(═O)—, —C(═O)— and —S(═O)—. Examples of —(C₃-C₁₂)-heterocycloalkyl groups are tetrahydrothiophenyl, tetrahydrofuryl, tetrahydropyranyl and dioxanyl.

In the context of the present invention, the expression “—(C₆-C₂₀)-aryl and —(C₆-C₂O-aryl-(C₆-C₂₀)-aryl-” encompasses mono- or polycyclic aromatic hydrocarbyl radicals. These have 6 to 20 ring atoms, more preferably 6 to 14 ring atoms, especially 6 to 10 ring atoms. Aryl is preferably —(C₆-C₁₀)-aryl and —(C₆-C₁₀)-aryl-(C₆-C₁₀)-aryl-. Aryl is especially phenyl, naphthyl, indenyl, fluorenyl, anthracenyl, phenanthrenyl, naphthacenyl, chrysenyl, pyrenyl, coronenyl. More particularly, aryl is phenyl, naphthyl and anthracenyl.

Substituted —(C₆-C₂₀)-aryl groups and —(C₆-C₂₀)-aryl-(C₆-C₂₀)-aryl groups may have one or more (e.g. 1, 2, 3, 4 or 5) substituents, depending on the ring size. These substituents are preferably each independently selected from —H, —(C₁-C₁₂)-alkyl, —O—(C₁-C₁₂)-alkyl, —O—(C₆-C₂₀)-aryl, —(C₆-C₂₀)-aryl, -halogen (such as Cl, F, Br, I), —COO—(C₁-C₁₂)-alkyl, —CONH—(C₁-C₁₂)-alkyl, —(C₆-C₂₀)-aryl-CONRC₁-C₁₂)-alkyl, —CO—(C₁-C₁₂)-alkyl, -CO-(C₆-C₂₀)-aryl, —COOH, —OH, —SO₃H, —SO₃Na, —NO₂, —CN, —NH₂, —N[(C₁-C₁₂)-alkyl]₂.

Substituted —(C₆-C₂₀)-aryl groups and —(C₆-C₂₀)-aryl-(C₆-C₂₀)-aryl groups are preferably substituted —(C₆-C₁₀)-aryl groups and —(C₆-C₁₀)-aryl-(C₆-C₁₀)-aryl groups, especially substituted phenyl or substituted naphthyl or substituted anthracenyl. Substituted —(C₆-C₂₀)-aryl groups preferably bear one or more, for example 1, 2, 3, 4 or 5, substituents selected from -(C₁-C₁₂)-alkyl groups, —(C₁-C₁₂)-alkoxy groups.

In one embodiment, R¹, R², R³, R⁴, R⁵, R⁶, R⁷, R⁸ are selected from:

—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —CO—(C₁-C₁₂)-alkyl, —CO—(C₆-C₂₀)-aryl, —N[(C₁-C₁₂)-alkyl]₂.

In one embodiment, R⁸, R⁹, R¹⁰, R¹¹, R¹², R¹³, R¹⁴, R¹⁵, R¹⁶, R¹⁷, R¹⁸ are selected from:

—H, —(C₁-C₁₂)-alkyl, —O—(C₁-C₁₂)-alkyl, —O—(C₆-C₂ₒ)-aryl, —(C₆-C₂₀)-aryl, —S-alkyl, —S-aryl, halogen, —CO—(C₁-C₁₂)-alkyl, —CO—(C₆-C₂₀)-aryl, —N[(C₁-C₁₂)-alkyl]₂.

In one embodiment, R¹, R², R³, R⁴, R⁵, R⁶, R⁷, R⁸ are selected from: —H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen.

In one embodiment, R⁹, R¹⁰, R¹¹, R¹², R¹³, R¹⁴, R¹⁵, R¹⁶, R¹⁷, R¹⁸ are selected from:

—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen.

In one embodiment, R¹, R², R³, R⁴, R⁵, R⁶, R⁷, R⁸ are selected from:

—H, —(C₁-C₁₂)-alkyl, —O—(C₁-C₁₂)-alkyl, —O—(C₆-C₂₀)-aryl, —(C₁-C₂₀)-aryl, —S-alkyl, —S-aryl.

In one embodiment, R⁹, R¹⁰, R¹¹, R¹², R¹³, R¹⁴, R¹⁵, R¹⁶, R¹⁷, R¹⁸ are selected from:

—H, —(C₁-C₁₂)-alkyl, —O—(C₁-C₁₂)-alkyl, —O—(C₆-C₂₀)-aryl, —(C₆-C₂₀)-aryl, -S-alkyl, —S-aryl.

In one embodiment, R¹, R², R³, R⁴, R⁵, R⁶, R⁷, R⁸ are selected from:

—H, —(C₁-C₁₂)-alkyl, —O—(C₁-C₁₂)-alkyl, —O—(C₆-C₂₀)-aryl.

R⁹, R¹⁰, R¹¹, R¹², R¹³, R¹⁴, R¹⁵, R¹⁶, R¹⁷, R¹⁸ are selected from:

—H, —(C₁-C₁₂)-alkyl, —O—(C₁-C₁₂)-alkyl, —O—(C₆-C₂₀)-aryl.

In one embodiment, R¹, R², R³, R⁴, R⁵, R⁶, R⁷, R⁸ are selected from:

—H, —(C₁-C₁₂)-alkyl, —O—(C₁-C₁₂)-alkyl.

In one embodiment, R⁹, R¹⁰, R¹¹, R¹², R¹³, R¹⁴, R¹⁵, R¹⁶, R¹⁷, R¹⁸ are selected from:

—H, —(C₁-C₁₂)-alkyl, —O—(C₁-C₁₂)-alkyl.

In one embodiment, the compound has one of the following structures (1) to (10).

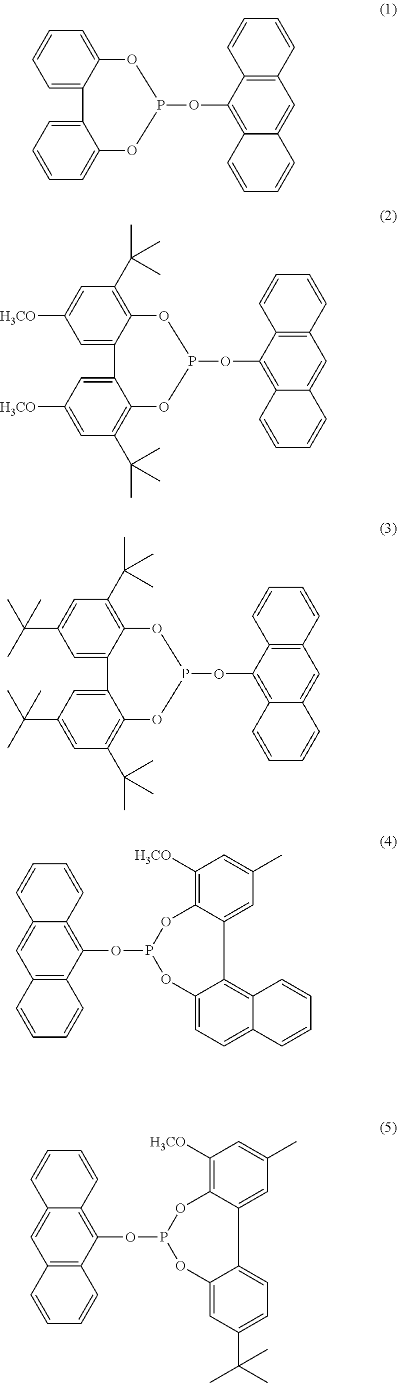

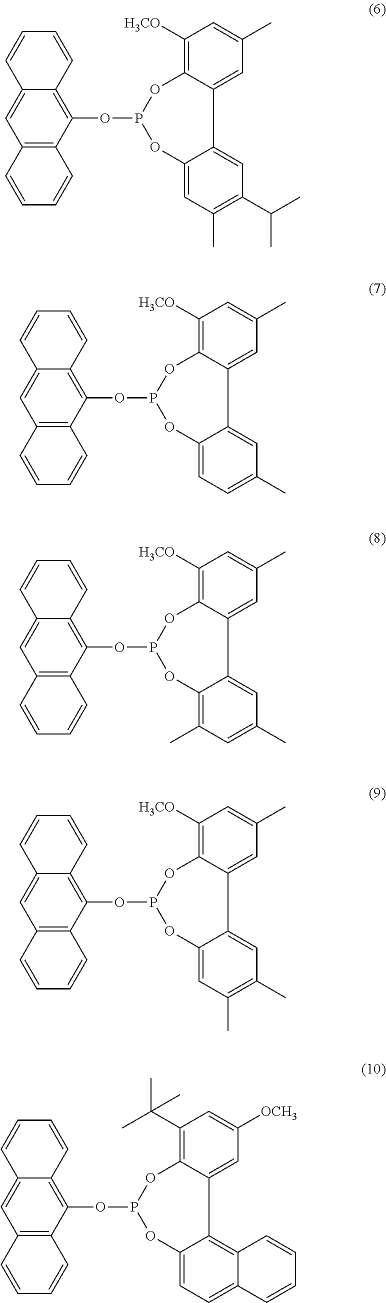 [SMILES: COc1cc(C)cc2c1op(Oc1c3ccccc3cc3ccccc13)oc1cc(C)c(C(C)C)cc12 ; COc1cc(C)cc2c1op(Oc1c3ccccc3cc3ccccc13)oc1ccc(C)cc12 ; COc1cc(C)cc2c1op(Oc1c3ccccc3cc3ccccc13)oc1c(C)cc(C)cc12 ; COc1cc(C)cc2c1op(Oc1c3ccccc3cc3ccccc13)oc1cc(C)c(C)cc12 ; COc1cc(C(C)(C)C)c2op(Oc3c4ccccc4cc4ccccc34)oc3ccc4ccccc4c3c2c1]

In one embodiment, the compound has the general structures I.

In one embodiment, the compound has the general structures II.

As well as the compounds, also claimed is a complex comprising these compounds.

Complex comprising:
a compound described above,
a metal atom selected from: Rh, Ru, Co, Ir.

In a preferred embodiment, the metal is Rh.

In this regard, see R. Franke, D. Selent, A. Bömer, “Applied Hydroformylation”, Chem. Rev., 2012, DOI:10.1021/cr3001803; p. 5688 Scheme 12 “General Method for the Preparation of a P-Modified Rh precatalyst” and references cited therein, and also P. W. N. M. van Leeuwen, in Rhodium Catalyzed Hydroformylation, P. W. N. M. van Leeuwen, C. Claver (eds.), Kluwer, Dordrecht, 2000, including p. 48 ff, p.233 ff., and references cited therein, and also K. D. Wiese and D. Obst in Top. Organomet. Chem. 2006, 18, 1-13; Springer Verlag Berlin Heidelberg 2006 p. 6 ff., and also references cited therein.

Additionally claimed is the use of the compound as ligand in a ligand-metal complex for catalysis of a hydroformylation reaction.

Use of a compound described above in a ligand-metal complex for catalysis of a hydroformylation reaction.

The process in which the compound is used as ligand in a ligand-metal complex for conversion of an olefin to an aldehyde is likewise claimed.

A process comprising the following process steps:

a) initially charging an olefin,

b) adding an above-described complex,

or an above-described compound and a substance including a metal atom selected from: Rh, Ru, Co, Ir,

c) feeding in H₂ and CO,

d) heating the reaction mixture, with conversion of the olefin to an aldehyde.

In this process, process steps a) to d) can be effected in any desired sequence.

In a preferred variant of the process, the metal atom is Rh.

An excess of ligands can also be used in this case and each ligand is not necessarily present bound in the form of a ligand-metal complex but is present as free ligand in the reaction mixture.

The reaction is conducted under customary conditions.

Preference is given to a temperature of 80° C. to 200° C. and a pressure of 1 bar to 300 bar.

Particular preference is given to a temperature of 100° C. to 160° C. and a pressure of 15 bar to 250 bar.

The reactants for the hydroformylation in the process of the invention are olefins or mixtures of olefins, especially monoolefins having 2 to 24, preferably 3 to 16 and more preferably 3 to 12 carbon atoms, having terminal or internal C—C double bonds, for example 1-propene, 1- or 2-pentene, 2-methyl-l-butene, 2-methyl-2-butene, 3-methyl-l-butene, 1-, 2- or 3-hexene, the C₆ olefin mixture obtained in the dimerization of propene (dipropene), heptenes, 2- or 3-methyl-1-hexenes, octenes, 2-methylheptenes, 3-methylheptenes, 5-methyl- 2-heptene, 6-methyl-2-heptene, 2-ethyl-l-hexene, the C₈ olefin mixture obtained in the dimerization of butenes (dibutene), nonenes, 2- or 3-methyloctenes, the C₉ olefin mixture obtained in the trimerization of propene (tripropene), decenes, 2-ethyl-1-octene, dodecenes, the C₁₂ olefin mixture obtained in the tetramerization or the trimerization of butenes (tetrapropene or tributene), tetradecenes, hexadecenes, the C₁₆ olefin mixture obtained in the tetramerization of butenes (tetrabutane), and olefin mixtures prepared by cooligomerization of olefins having different numbers of carbon atoms (preferably 2 to 4).

Having generally described this invention, a further understanding can be obtained by reference to certain specific examples which are provided herein for purposes of illustration only, and are not intended to be limiting unless otherwise specified.

The invention is illustrated in detail hereinafter by working examples.

### EXAMPLES

### General Operating Procedures

All the preparations which follow were carried out under protective gas using standard Schlenk techniques. The solvents were dried over suitable desiccants before use (Purification of Laboratory Chemicals, W. L. F. Armarego (Author), Christina Chai (Author), Butterworth Heinemann (Elsevier), 6th edition, Oxford 2009).

Phosphorus trichloride (Aldrich) was distilled under argon before use. All preparative operations were effected in baked-out vessels. The products were characterized by means of NMR spectroscopy. Chemical shifts (δ) are reported in ppm. The ³¹P NMR signals were referenced according to: SR_{31P}=SR_{1H}*(BF_{31P}/BF_{1H})=SR_{1H}* 0.4048. (Robin K. Harris, Edwin D. Becker, Sonia M. Cabral de Menezes, Robin Goodfellow, and Pierre Granger, Pure Appl. Chem., 2001, 73, 1795-1818; Robin K. Harris, Edwin D. Becker, Sonia M. Cabral de Menezes, Pierre Granger, Roy E. Hoffman and Kurt W. Zilm, Pure Appl. Chem., 2008, 80, 59-84).

The recording of nuclear resonance spectra was effected on Bruker Avance 300 or Bruker Avance 400, gas chromatography analysis on Agilent GC 7890A, elemental analysis on Leco TruSpec CHNS and Varian ICP-OES 715, and ESI-TOF mass spectrometry on Thermo Electron Finnigan MAT 95-XP and Agilent 6890 N/5973 instruments.

### Biphenols

The biphenols are synthesized analogously to DE102013203865 and DE102013203867.

### Synthesis of the Chlorophosphites

The synthesis of the mono- and dichlorophosphites such as (anthracen-9-yloxy)-dichlorophosphine, 2,6-diphenylphenoxydichlorophosphine or 6-chlorodibenzo[d,f][1,3,2]-dioxaphosphepine is known to a person skilled in the art and is carried out in a known manner. Chlorophosphites can be prepared by addition of phosphorus trichlorite in the presence of a base. For detailed information, see also “Phosphorous(III) Ligands in Homogeneous Catalysis—Design and Synthesis” by Paul C. J. Kamer and Piet W. N. M. van Leeuwen; John Wiley and Sons, 2012; including p. 94 ff. and references cited therein.

### Synthesis of the Monophosphites

### 6-(Anthracen-9-yloxy)dibenzo[d,f][1,3,2]dioxaphosphepine

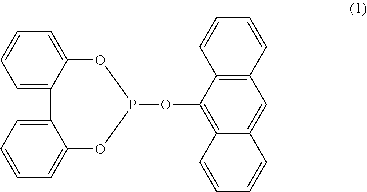 [SMILES: c1ccc2c(Op3oc4ccccc4c4ccccc4o3)c3ccccc3cc2c1]

To a stirred solution of anthracen-9-ol (0.389 g; 2.00 mmol) in toluene (9 ml) was added triethylamine (1.858 g; 18.36 mmol) and the resulting mixture added dropwise at 0° C. to a solution of 6-chlorodibenzo[d,f][1,3,2]dioxaphosphepine (0.501 g; 2.00 mmol) in toluene (9 ml). The reaction mixture was stirred overnight and filtered. The filtrate was concentrated to dryness under vacuum. The resulting solid was dried at 40° C. for 2 h and then recrystallized from hot dichloromethane (3 ml). Yield: 0.206 g (0.504 mmol; 25%).

Elemental analysis (calculated for C₂₆H₁₇O₃P=408.37 g/mol) C 76.59 (76.47); H 4.14 (4.20); P 7.64 (7.58)%.

³¹P-NMR (CD₂Cl₂): 148.4 ppm.

¹H-NMR (CD₂Cl₂): 7.37-7.72 (m, 12H); 8.10 (m, 2H); 8.40 (s, 1H); 8.56 (m, 2H) ppm.

¹³C-NMR (CD₂Cl₂): 122.6; 122.9; 123.7; 125.0; 126.1; 126.2; 126.4; 128.6; 129.8; 130.6; 131.5; 132.5; 143.4 (d, J_{CP}=6.0 Hz); 149.4 (d, J_{CP}=5.8 Hz) ppm.

ESI-TOF/HRMS: m/e 409.09945 (M+H)⁺.

### 6-(Anthracen-9-yloxy)-4,8-di-tert-butyl-2,10-dimethoxydibenzo[d,f][1,3,2]dioxaphosphepine

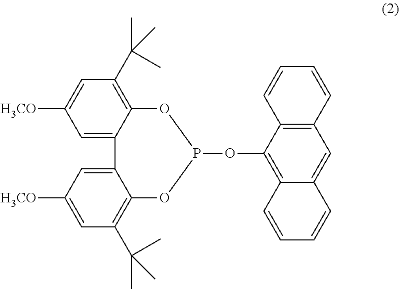 [SMILES: COc1cc(C(C)(C)C)c2op(Oc3c4ccccc4cc4ccccc34)oc3c(C(C)(C)C)cc(OC)cc3c2c1]

To a stirred solution of anthracen-9-ol (0.388 g; 2 mmol) in toluene (9 ml) was added triethylamine (1.854 g; 18.33 mmol) and the resulting mixture added dropwise at 0° C. to a solution of 4,8-di-tert-butyl-6-chloro-2,10-dimethoxydibenzo[d,f][1,3,2]dioxaphosphepine (0.845 g; 2 mmol) in toluene (8 ml). The reaction mixture was stirred overnight and filtered. The filtrate is concentrated to dryness under vacuum. The residue obtained was stirred twice with hexane (4 ml each) and filtered and then dried under vacuum. Yield: 0.206 g (0.355 mmol; 18%).

Elemental analysis (calculated for C₃₆H₃₇O₅P=580.63 g/mol) C 74.28 (74.46); H 6.62 (6.42); P 5.39 (5.33)%.

³¹P-NMR (CD₂Cl₂): 140.4 ppm.

¹H-NMR (CD₂Cl₂): 1.39 (s, 18H); 3.92 (s, 6H); 6.93 (d, ⁵J_{HH}=3.1 Hz, 2H, Hₐᵣₒₘ); 7.14 (d,⁵J_{HH}=3.1 Hz, 2H, Hₐᵣₒₘ); 7.51 (m, 4H, Hₐᵣₒₘ); 8.05 (m, 2H, Hₐᵣₒₘ); 8.34 (s, 1H, Hₐᵣₒₘ); 8.41 (m, 2H, Hₐᵣₒₘ) ppm.

¹³C-NMR (CD₂Cl₂): 31.0; 35.7; 56.0; 113.5; 114.9; 123.2; 123.7 (d, J_{CP}=5.4 Hz); 124.8; 125.9; 126.0; 128.4; 132.6; 134.2 (d, J_{CP}=3.9 Hz); 141.7 (d, J_{CP}=6.0 Hz); 143.4;

143.5; 156.6 ppm.

ESI-TOF/HRMS: m/e 581.24490 (M+H)⁺.

### 6-(Anthracen-9-yloxy)-2,4,8,10-tetra-tert-butyldibenzo[d,f][1,3,2]dioxaphosphepine

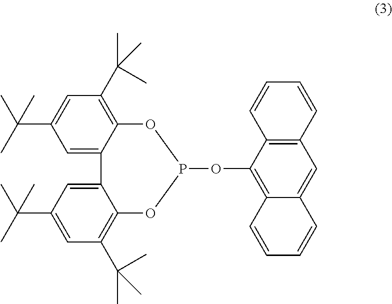 [SMILES: CC(C)(C)c1cc(C(C)(C)C)c2op(Oc3c4ccccc4cc4ccccc34)oc3c(C(C)(C)C)cc(C(C)(C)C)cc3c2c1]

To a stirred suspension of anthracen-9-ol (0.281 g; 1.45 mmol) in toluene (7 ml) was added triethylamine (1.344 g; 13.28 mmol) and the resulting mixture added dropwise at 0° C. to a solution of 2,4,8,10-tetra-tert-butyl-6-chlorodibenzo [d,f][1,3,2]dioxaphosphepine (0.724 g; 1.52 mmol) in toluene (6 ml). The reaction mixture was stirred overnight and filtered. The filtrate was concentrated to dryness under vacuum and the resulting residue recrystallized from hexane (4 ml). Yield: 0.559 g (0.883 mmol; 61%).

Elemental analysis (calculated for C₄₂H₄₉O₃P=632.78 g/mol) C 79.83 (79.71); H 7.61 (7.81); P 5.01 (4.89)%.

³¹P-NMR (CD₂Cl₂): 140.7 ppm.

¹H-NMR (CD₂Cl₂): 1.41 (s, 18H C(CH₃)₃); 1.50 (s, 18H C(CH₃)₃); 7.40 (d, ⁵J_{HH}=2.4 Hz, 2H, Hₐᵣₒₘ); 7.43-7.56 (m, 4H, Hₐᵣₒₘ); 7.60 (d, ⁵J_{HH}=2.4 Hz, 2H, Hₐᵣₒₘ); 8.05 (m, 2H, Hₐᵣₒₘ); 8.34 (s, 1H, Hₐᵣₒₘ); 8.38 (m, 2H, Hₐᵣₒₘ) ppm.

¹³C-NMR (CD₂Cl₂): 31.2; 31.2; 31.8; 35.1; 35.7; 123.1; 123.7 (d, J_{CP}=5.3 Hz); 124.8; 125.0; 125.8; 126.0; 127.2; 128.4; 132.6; 133.3 (d, J_{CP}=3.7 Hz); 141.0; 143.5; 145.8 (d, J_{CP}=6.2 Hz); 147.7 ppm.

ESI-TOF/HRMS: m/e 633.34934 (M+H)⁺.

### 6-(Anthracen-9-yloxy)-4-methoxy-2-methylbenzo[d]naphtho[1,2-f][1,3,2]-dioxaphosphepine

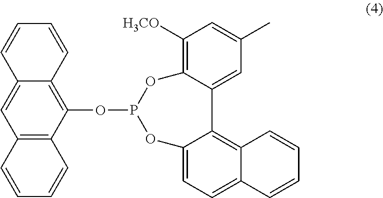 [SMILES: COc1cc(C)cc2c1op(Oc1c3ccccc3cc3ccccc13)oc1ccc3ccccc3c12]

To a solution of 1-(2-hydroxy-3-methoxy-5-methylphenyl)naphthalen-2-ol (0.448 g; 1.598 mmol) in THF (8 ml) was added pyridine (0.284 g; 3.596 mmol). A solution of (anthracen-9-yloxy)dichlorophosphine (0.472 g; 1.598 mmol) in THF (6 ml) was then added dropwise at 0° C. The reaction mixture was stirred overnight, filtered, and the filtrate was concentrated to dryness under vacuum. The residue obtained was recrystallized from hot dichloromethane (13 ml). Yield: 0.302 g (0.601 mmol; 38%).

Elemental analysis (calculated for C₃₂H₂₃O₄P=502.50 g/mol) C 76.38 (76.49); H 4.68 (4.61); P 6.15 (6.16)%.

³¹P-NMR (THF-d₈): 147.3; 152.7 ppm.

¹H-NMR (THF-d₈): 29-2.31 (2s, 3H); 3.77-3.87 (2s, 3H); 6.92 (m, 1H, Hₐᵣₒₘ); 7.01 (m, 1H, Hₐᵣₒₘ); 7.34-7.46 (m, 7H, Hₐᵣₒₘ); 7.47-7.58 (m, 1H, H_{awn}); 7.81-7.95 (m, 4H, Hₐᵣₒₘ); 8.11 (m, 1H, Hₐᵣₒₘ); 8.25 (m, 1H, Hₐᵣₒₘ); 8.47 (m, 1H, Hₐᵣₒₘ); 8.74 (m, 1H, Hₐᵣₒₘ) ppm.

¹³C-NMR (THF-d₈): 20.6; 20.7; 55.2; 55.5; 112.4; 112.8; 121.4; 121.6; 122.6; 123.0; 123.0; 123.8; 124.0; 124.6; 124.6; 124.9; 125.1; 125.5; 125.5; 125.6; 125.7; 125.7; 126.2; 126.2; 126.6; 126.7; 127.9; 128.0; 128.4; 128.4; 128.5; 128.5; 129.5; 130.0; 130.2 (d, J_{CP}=4.3 Hz); 132.0; 132.3; 132.5; 134.0; 134.8; 135.7; 137.6; 143.2 (d, J_{CP}=7.7 Hz); 143.5 (d, J_{CP}=8.3 Hz); 145.8 (d, J_{CP}=2.8 Hz); 147.2 (d, J_{CP}=6.7 Hz); 152.2; 152.5 (d, J_{CP}=2.6 Hz) ppm.

ESI-TOF/HRMS: m/e 503.14057 (M+H)⁺.

### 6-(Anthracen-9-yloxy)-9-(tert-butyl)-4-methoxy-2-methyldibenzo[d,f][1,3,2]-dioxaphosphepine

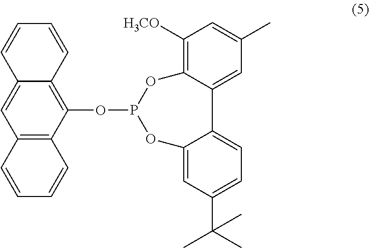 [SMILES: COc1cc(C)cc2c1op(Oc1c3ccccc3cc3ccccc13)oc1cc(C(C)(C)C)ccc12]

To a stirred suspension of 4′-(tert-butyl)-3-methoxy-5-methyl41,1′-biphenyl]-2,2′-diol (0.703 g; 2.46 mmol) in toluene (7 ml) was added triethylamine (2.277 g; 22.50 mmol) and this mixture added dropwise at 0° C. to a solution of (anthracen-9-yloxy)dichlorophosphine (0.725 g; 2.46 mmol) in toluene (13 ml). The reaction mixture was stirred overnight and then filtered. The filtrate was concentrated to dryness under vacuum and the resulting residue recrystallized from toluene (2 m1). Yield: 0.320 g (0.630 mmol; 26%). Elemental analysis (calculated for C₃₂H₂₉O₄P=508.55 g/mol) C 75.48 (75.58); H 5.86 (5.75); P 6.03 (6.09)%.

³¹P-NMR (CD₂Cl₂): 148.4 ppm.

¹H-NMR (CD₂Cl₂): 44 (s, 9H); 2.49 (m, 3H); 4.00 (s, 3H); 6.94-7.04 (m, 2H, Hₐᵣₒₘ); 7.46 (m, 2H, Hₐᵣₒₘ); 7.56-7.69 (m, 5H, Hₐᵣₒₘ); 8.10 (m, 2H, Hₐᵣₒₘ); 8.39 (m, 1H, Hₐᵣₒₘ); 8.78 (m, 2H, Hₐᵣₒₘ) ppm.

¹³C-NMR (CD₂Cl₂): 21.7; 31.3; 31.4; 35.1; 56.5; 113.0; 119.5; 121.9; 123.2; 123.3; 123.5; 125.2 (d, J_{CP}=5.4 Hz); 126.3 (d, J_{CP}=9.5 Hz); 128.5; 129.9; 132.0 (d, J_{CP}=3.0 Hz); 132.6; 136.1; 136.2 (d, J_{CP}=6.0 Hz); 143.9 (d, J_{CP}=6.7 Hz); 149.3 (d, J_{CP}=5.7 Hz); 152.1; 153.8 ppm.

ESI-TOF/HRMS: m/e 509.18744 (M+H)⁺.

### 6-(Anthracen-9-yloxy)-2-isopropyl-8-methoxy-3,10-dimethyldibenzo[d,f][1,3,2]-dioxaphosphepine

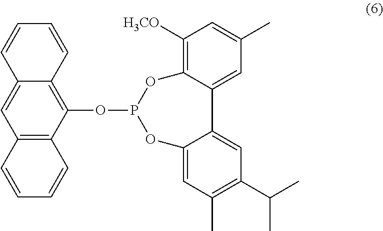 [SMILES: COc1cc(C)cc2c1op(Oc1c3ccccc3cc3ccccc13)oc1cc(C)c(C(C)C)cc12]

To a solution of 5′-isopropyl-3-methoxy-4′,5-dimethyl-[1,1′-biphenyl]-2,2′-diol (0.359 g; 1.252 mmol) in toluene (7 ml) was added triethylamine (1.161 g; 11.473 mmol), cooled to 0° C. and to this mixture was added dropwise a solution of (anthracen-9-yloxy)dichlorophosphine (0.370 g; 1.252 mmol) in toluene (9 ml). The reaction mixture was stirred overnight and then filtered. The filtrate was concentrated to dryness under vacuum. Yield: 0.594 g (1.168 mmol; 93%).

Elemental analysis (calculated for C₃₂H₂₉O₄P=508.55 g/mol) C 75.46 (75.58); H 5.90 (5.75); P 6.09 (6.09)%.

³¹P-NMR (CD₂Cl₂): 148.5 ppm.

¹H-NMR (CD₂Cl₂): 38 (d, ²J_{HH}=6.8 Hz, 3H); 1.39 (d, ²J_{CP}=6.8 Hz, 3H); 2.46 (s, 3H); 2.52 (s, 3H); 3.22-3.32 (m, 1H); 3.99 (s, 3H); 6.95 (m, 1H, Hₐᵣₒₘ); 7.07 (m, 1H, Hₐᵣₒₘ); 7.20 (m, 1H, Hₐᵣₒₘ); 7.52 (s, 1H, Hₐᵣₒₘ); 7.57-7.68 (m, 4H, Hₐᵣₒₘ); 8.10 (m, 2H, Hₐᵣₒₘ); 8.39 (m, 1H, Hₐᵣₒₘ); 8.79 (m, 2H, Hₐᵣₒₘ) ppm.

¹³C-NMR (CD₂Cl₂): 19.3; 21.7; 23.6; 23.6; 29.5; 56.5; 113.0; 121.9; 123.4; 123.5; 123.8; 125.2; (d, J_{CP}=3.3 Hz); 125.7; 126.3 (d, J_{CP}=3.3 Hz); 126.7; 128.6 (d, J_{CP}=8.7 Hz); 129.4; 132.5 (d, J_{CP}=3.6 Hz); 132.6; 136.1; 136.2 (d, J_{CP}=6.2 Hz); 137.5; 138.4; 143.9 (d, J_{CP}=6.5 Hz); 144.8; 146.9 (d, J_{CP}=5.9 Hz); 152.1 ppm.

ESI-TOF/HRMS: m/e 509.18763 (M+H)⁺.

### 6-(Anthracen-9-yloxy)-4-methoxy-2,10-dimethyldibenzo[d,f][1,3,2]dioxaphosphepine

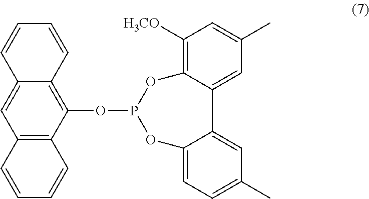 [SMILES: COc1cc(C)cc2c1op(Oc1c3ccccc3cc3ccccc13)oc1ccc(C)cc12]

To a stirred solution of 3-methoxy-5,5’-dimethyl-[1,1′-biphenyl]-2,2′-diol (0.655 g; 2.68 mmol) in toluene (16 ml) was added triethylamine (2.487 g; 24.58 mmol) and the mixture cooled to 0° C. To this mixture was added dropwise a solution of (anthracen-9-yloxy)dichlorophosphine (0.792 g; 2.683 mmol) in toluene (18 ml). The reaction mixture was stirred overnight and then filtered. The filtrate was concentrated to dryness under vacuum and the residue dried at 50° C./0.1 mbar for 3 h. Yield: 1.020 g (2.187 mmol; 82%). Elemental analysis (calculated for C₂₉H₂₃O₄P=466.47 g/mol) C 74.58 (74.67); H 5.19 (4.97); P 6.78 (6.64)%.

³¹P-NMR (CD₂Cl₂): 148.6 ppm.

¹H-NMR (CD₂Cl₂): 2.51 (m, 6H); 3.99 (s, 3H); 6.96 (m, 1H, Hₐᵣₒₘ); 7.06 (m, 1H, Hₐᵣₒₘ); 7.32-7.37 (m, 2H, Hₐᵣₒₘ); 7.48 (m, 1H, Hₐᵣₒₘ); 7.57-7.69 (m, 4H, Hₐᵣₒₘ); 8.10 (m, 2H, Hₐᵣₒₘ); 8.39 (m, 1H, Hₐᵣₒₘ); 8.79 (m, 2H, Hₐᵣₒₘ) ppm.

¹³C-NMR (CD₂Cl₂): 21.1; 21.7; 56.4; 113.2; 122.0; 122.2; 123.3; 123.5; 125.1 (d, J_{CP}=3.7 Hz); 126.2; 126.3; 128.5; 130.3; 130.9; 131.3 (d, J_{CP}=3.2 Hz); 132.1 (d, J_{CP}=3.3 Hz); 132.5 (d, J_{CP}=2.0 Hz); 135.7; 136.1; 136.3 (d, J_{CP}=5.8 Hz); 143.9 (d, J_{CP}=7.1 Hz); 147.3 (d, J_{CP}=5.4 Hz); 152.0 (d, J_{CP}=2.0 Hz) ppm.

ESI-TOF/HRMS: m/e 467.10093 (M+H)⁺.

### 6-(Anthracen-9-yloxy)-4-methoxy-2,8,10-trimethyldibenzo[d,f][1,3,2]dioxaphosphepine

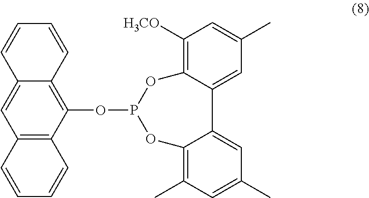 [SMILES: COc1cc(C)cc2c1op(Oc1c3ccccc3cc3ccccc13)oc1c(C)cc(C)cc12]

To a solution of 3-methoxy-3′,5,5′-trimethyl-[1,1′-biphenyl]-2,2′-diol (0.409 g; 1.58 mmol) in toluene (9 ml) was added triethylamine (2.468 g; 14.51 mmol), cooled to 0° C. and to this mixture was added dropwise a solution of (anthracen-9-yloxy)dichlorophosphine (0.467 g; 1.584 mmol) in toluene (11 ml). The reaction mixture was stirred overnight and then filtered. The filtrate was concentrated to dryness under vacuum and the resulting residue recrystallized from hexane (17 ml). Yield: 0.511 g (1.063 mmol; 67%). Elemental analysis (calculated for C₃₀H₂₅O₄P=480.50 g/ mol) C 74.53 (74.99); H 5.53 (5.24); P 6.48 (6.45)%.

³¹P-NMR (CD₂Cl₂): 147.7 ppm.

¹H-NMR (CD₂Cl₂): 2.46-2.50 (m, 9H); 4.02 (s, 3H); 6.96 (m, 1H, Hₐᵣₒₘ); 7.05 (m, 1H, Hₐᵣₒₘ); 7.20 (m, 1H, Hₐᵣₒₘ); 7.30 (m, 1H, Hₐᵣₒₘ); 7.56-7.68 (m, 4H, Hₐᵣₒₘ); 8.10 (m, 2H, Hₐᵣₒₘ); 8.40 (m, 1H, Hₐᵣₒₘ); 8.88 (m, 2H, Hₐᵣₒₘ) ppm.

¹³C-NMR (CD₂Cl₂): 17.0; 21.1; 21.7; 56.3; 113.0; 122.2; 123.4; 123.5; 125.3 (d, J_{CP}=3.6 Hz); 125.7; 126.3 (d, J_{CP}=5.5 Hz); 128.4; 128.5; 130.9; 131.6 (d, J_{CP}=3.7 Hz); 132.0; 132.0; 132.5; 135.4; 135.8; 136.8 (d, J_{CP}=8.0 Hz); 143.9 (d, J_{CP}=7.4 Hz); 145.3 (d, J_{CP}=4.0 Hz); 151.9 ppm. ESI-TOF/HRMS: m/e 481.15626 (M+H)⁺.

### 6-(Anthraces-9-yloxy)-8-methoxy-2,3,10-trimethyldibenzo[d,f][1,3,2]dioxaphosphepine

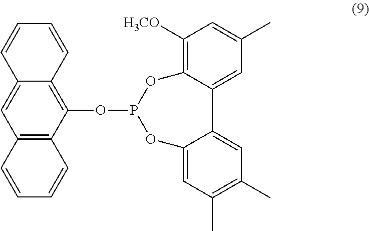 [SMILES: COc1cc(C)cc2c1op(Oc1c3ccccc3cc3ccccc13)oc1cc(C)c(C)cc12]

To a solution of 3-methoxy-4′,5,5′-trimethyl-[1,1′-biphenyl]-2,2′-diol (0.518 g; 2.00 mmol) in toluene (11 ml) was added triethylamine (1.857 g; 18.35 mmol) and the mixture cooled to 0° C. To this mixture was added dropwise a solution of (anthracen-9-yloxy)dichlorophosphine (0.591 g; 2.00 mmol) in toluene (8 ml). After addition of further toluene (30 ml), the reaction mixture was stirred overnight. The mixture was then filtered and the solvent was removed under vacuum. The residue obtained was taken up in toluene (11 ml) and the resulting suspension gently heated and then filtered. The filtrate was concentrated to dryness under vacuum and the residue obtained dried at 50° C./0.1 mbar. Yield: 0.489 g (1.018 mmol; 51%).

Elemental analysis (calculated for C₃₀H₂₅O₄P=480.50 g/mol) C 74.77 (74.99); H 5.10 (5.24); P 6.47 (6.45)%.

³¹P-NMR (CD₂Cl₂): 148.2 ppm.

¹H-NMR (CD₂Cl₂): 2.38 (m, 6H); 2.49 (m, 3H); 3.98 (s, 3H); 6.93 (m, 1H, Hₐᵣₒₘ); 7.03 (m, 1H, Hₐᵣₒₘ); 7.19 (m, 1H, Hₐᵣₒₘ); 7.40 (m, 1H, Hₐᵣₒₘ); 7.56-7.68 (m, 4H, Hₐᵣₒₘ); 8.09 (m, 2H, Hₐᵣₒₘ); 8.38 (m, 1H, Hₐᵣₒₘ); 8.76 (m, 2H, Hₐᵣₒₘ) ppm.

¹³C-NMR (CD₂Cl₂): 19.4; 19.8; 21.7; 56.4; 112.9; 121.8; 123.3; 123.3; 123.4; 125.1 (d, J_{CP}=3.5 Hz); 125.6 (d, J_{CP}=3.5 Hz); 126.1; 126.2; 128.4; 131.1; 132.1 (d, J_{CP}=3.1 Hz); 132.5; 134.4; 136.0; 136.2 (d, J_{CP}=6.0 Hz); 138.8; 143.9 (d, J_{CP}=6.9 Hz); 147.2 (d, J_{CP}=5.7 Hz); 152.0 ppm. ESI-TOF/HRMS: m/e 481.15601 (M+H)⁺.

### 6-(Anthracen-9-yloxy)-4-(tert-butyl)-2-methoxybenzo[d]naphtho[1,2-f][1,3,2]-dioxaphosphepine

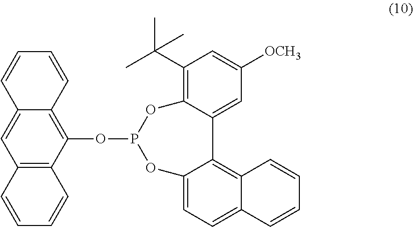 [SMILES: COc1cc(C(C)(C)C)c2op(Oc3c4ccccc4cc4ccccc34)oc3ccc4ccccc4c3c2c1]

To a solution of 1-(3-(tert-butyl)-2-hydroxy-5-methoxyphenyl)naphthalen-2-ol (0.730 g; 2.26 mmol) in toluene (14 ml) was added triethylamine (2.097 g; 20.73 mmol) and to this mixture at 0° C. was added dropwise a solution of (anthracen-9-yloxy)dichlorophosphine (0.668 g; 2.26 mmol) in toluene (10 ml). The reaction mixture was stirred overnight and then filtered. The filtrate was concentrated to dryness under vacuum and the resulting residue recrystallized from hexane (28 ml). Yield: 0.708 g (1.30 mmol; 58%). Elemental analysis (calculated for C₃₅H₂₉O₄P=544.58 g/mol) C 76.96 (77.19); H 5.38 (5.37); P 5.66 (5.69)%.

³¹P-NMR (CD₂Cl₂): 150.0 ppm.

¹H-NMR (CD₂Cl₂): 53 (s, 9H); 3.88 (s, 3H); 7.10 (d, J_{HH}=3.1 Hz, 1H, Hₐᵣₒₘ); 7.18 (d, J_{HH}=3.1 Hz, ¹H, Hₐᵣₒₘ); 7.54-7.64 (m, 6H, Hₐᵣₒₘ); 7.78 (m, 1H, Hₐᵣₒₘ); 7.99-8.10 (m, 4H, Hₐᵣₒₘ); 8.18-8.22 (m, 1H, Hₐᵣₒₘ); 8.38 (m, 1H, Hₐᵣₒₘ); 8.62 (m, 2H, Hₐᵣₒₘ) ppm.

¹³C-NMR (CD₂Cl₂): 31.2; 35.8; 56.1; 115.0; 115.1; 121.7; 123.1; 123.7; 125.0 (d, J_{CP}=3.7 Hz); 125.7; 126.0; 126.2; 126.4; 127.3; 128.6; 128.9; 129.4; 130.1; 130.7 (d, J_{CP}=5.0 Hz); 132.4; 132.5; 133.0; 138.4; 142.1; 143.7 (d, J_{CP}=7.2 Hz); 144.4; 146.8 (d, J_{CP}=5.5 Hz); 156.1 ppm. ESI-TOF/HRMS: m/e 545.18764 (M+H)⁺.

### 6-([1,1′:3′,1″-Terphenyl]-2′-yloxy)-4,8-di-tert-butyl-2,10-dimethoxydibenzo[d,f][1,3,2]-dioxaphosphepine

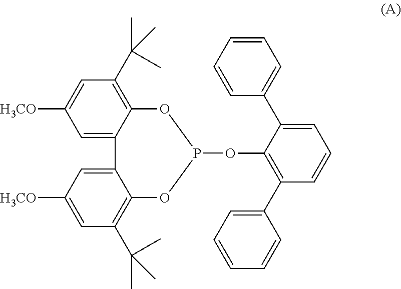 [SMILES: COc1cc(C(C)(C)C)c2op(Oc3c(-c4ccccc4)cccc3-c3ccccc3)oc3c(C(C)(C)C)cc(OC)cc3c2c1]

To a solution of 2,6-diphenylphenol (0.411 g; 1.65 mmol) in toluene (8 ml) was added triethylamine (1.529 g; 15.11 mmol) and the resulting mixture added dropwise at 0° C. to a solution of 4,8-di-tert-butyl-6-chloro-2,10-dimethoxydibenzo[d,f][1,3,2]dioxaphosphepine (0.697 g; 1.65 mmol) in toluene (6 ml). The reaction mixture was stirred overnight at room temperature and for 5 h at 70° C. The mixture was then filtered and filtrate was concentrated to dryness under vacuum. The residue obtained was recrystallized from hexane (4 ml). Yield: 0.417 g (0.659 mmol; 40%).

Elemental analysis (calculated for C₄₀H₄₁O₅P=632.70 g/mol) C 75.95 (75.93); H 6.52 (6.53); P 5.01 (5.00)%.

³¹P-NMR (CD₂Cl₂): ^{140.9} ppm.

¹H-NMR (CD₂Cl₂): 1.37 (s, 18H); 3.84 (s, 6H); 6.51 (d, ⁵J_{HH}=3.1 Hz, 2H, Hₐᵣₒₘ); 6.89-7.95 (m, br, 15H, Hₐᵣₒₘ) ppm.

¹³C-NMR (CD₂Cl₂): 31.1; 35.5; 55.9; 113.0; 114.5; 125.2; 126.5-131.0 (broad, overlapping signals); 133.8 (d, J_{CP}=4.0 Hz); 141.5 (d, J_{CP}=6.3 Hz); 142.6; 144.8; 156.1 ppm.

ESI-TOF/HRMS: m/e 633.27654 (M+H)⁺.

### 6-([1,1′:3′,1″-Terphenyl]-2′-yloxy)-2,4,8,10-tetra-tert-butyldibenzo[d,f][1,3,2]-dioxaphosphepine

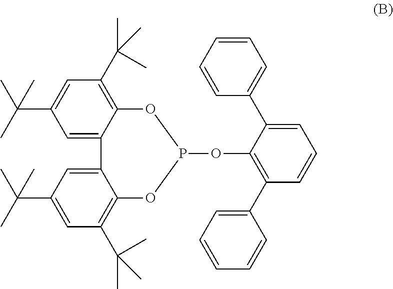 [SMILES: CC(C)(C)c1cc(C(C)(C)C)c2op(Oc3c(-c4ccccc4)cccc3-c3ccccc3)oc3c(C(C)(C)C)cc(C(C)(C)C)cc3c2c1]

To a solution of 3,3′,5,5′-tetra-tert-butyl-[1,1′-biphenyl]-2,2′-diol (0.616 g; 1.50 mmol) in toluene (13 ml) was added triethylamine (1.390 g; 13.74 mmol) and the mixture cooled to 0° C. To this mixture was added dropwise a solution of (2,6-diphenylphenoxy)dichlorophosphine (0.521 g; 1.50 mmol) in toluene (3 ml). The reaction mixture was stirred overnight and filtered. The filtrate was concentrated to dryness under vacuum and the resulting residue taken up in dichloromethane (3 ml) and filtered over silica gel. The filtrate was concentrated under vacuum and the solid obtained dried at 50° C./0.1 mbar. Yield: 0.955 g (1.394 mmol; 93%). Elemental analysis (calculated for C₄₆H₅₃O₃P=684.90 g/mol) C 80.60 (80.67); H 7.71 (7.80); P 4.36 (4.52)%.

³¹P-NMR (CD₂Cl₂): 138.7 ppm.

¹H-NMR (CD₂Cl₂): 1.29-1.46 (m, br, 36H C(CH₃)₃); 6.76-7.13 (m, br, 4H, Hₐᵣₒₘ); 7.30-7.34 (m, 1H, Hₐᵣₒₘ); 7.34-7.40 (m, 2H, Hₐᵣₒₘ)_{;} 7.40-7.87 (m, br, 10H, Hₐₘₘ) ppm.

¹³C-NMR (CD₂Cl₂): 31.3; 31.3; 31.7; 34.9; 35.6; 124.6; 125.2; 125.7; 126.7; 128.6; 129.4; 133.0; 137.7; 138.4; 140.5; 143.5; 144.9; 145.5; 146.9; 150.2; additional broad signals between 127 and 131 ppm.

ESI-TOF/HRMS: m/e 685.38089 (M+H)⁺.

### Procedure for the Catalyst Experiments

The hydroformylation was conducted in a 200 ml autoclave equipped with pressure-maintaining device, gas flow meter, sparging stirrer and pressure pipette from Premex Reactor AG, Lengau, Switzerland. To minimize the influence of moisture and oxygen, the toluene used as solvent was dried with sodium ketyl and distilled under argon. The olefin used was an octene mixture: n-octene (Oxeno GmbH, mixture of octene isomers of 1-octene: ˜3%; cis+trans-2-octene: ˜49%; cis+trans-3-octene: ˜29%; cis+trans-4-octene: ˜16%; structurally isomeric octenes: ˜3% were heated under reflux for several hours over sodium and distilled under argon.

For the experiments, the following solutions of rhodium in the form of [(acac)Rh(COD)] (acac=acetylacetonate anion; COD=1,5-cyclooctadiene, Umicore) as the catalyst precursor in toluene were introduced into the autoclave under an argon atmosphere: for experiments at 100 ppm by mass of rhodium 10 ml of a 4.31 millimolar solution, for 40 or 60 ppm by mass the same amount of an appropriately diluted solution. The appropriate amount of the phosphite compound (5 ligand equivalents per rhodium) dissolved in toluene was then added. By addition of further toluene (the total amount of toluene was determined for the GC analysis, see below), the initial volume of the catalyst solution was adjusted to 41.0 ml. The mass of toluene introduced was determined in each case. Weight of n-octene: 10.70 g (95.35 mmol). The autoclave was heated to the temperatures stated in each case at a total gas pressure (synthesis gas: Linde; H₂ (99.999%): CO (99.997%)=1:1) of a) 42 bar for a final pressure of 50 bar or b) 12 bar for a final pressure of 20 bar with stirring (1500 rpm). After reaching the reaction temperature, the synthesis gas pressure was increased to a) 48.5 bar for a final pressure of 50 bar or b) 19.5 bar for a final pressure of 20 bar and the reactant introduced under a positive pressure of ca. 3 bar set in the pressure pipette. The reaction was conducted at a constant pressure of 50 or 20 bar (closed-loop pressure controller from Bronkhorst, the Netherlands) respectively over 4 h. After the reaction time had elapsed, the autoclave was cooled to room temperature, decompressed while stirring and purged with argon. 1 ml of each reaction mixture was removed immediately after the stirrer had been switched off, diluted with 5 ml of pentane and analysed by gas chromatography: HP 5890 Series II plus, PONA, 50 mm×0.2 mm×0.5 The quantitative determination of residual olefins and aldehydes was carried out with reference to the solvent toluene as internal standard.

### Catalyst Results

The results of the catalyst experiments are summarized in Tables 1 and 2.

TABLE 1

| Ligand | p (bar) | T (° C.) | t (h) | [Rh] (ppm) | L/Rh | Yield (%) |
|---|---|---|---|---|---|---|
| 1* | 50 | 120 | 4 | 100 | 5 | 98 |
| 2* | 50 | 120 | 4 | 100 | 5 | 97 |
| 2* | 50 | 120 | 4 | 60 | 5 | 97 |
| 2* | 50 | 120 | 4 | 40 | 5 | 96 |
| 3* | 50 | 120 | 4 | 100 | 5 | 98 |
| 4* | 50 | 120 | 4 | 100 | 5 | 92 |
| 5* | 50 | 120 | 4 | 100 | 5 | 100 |
| 6* | 50 | 120 | 4 | 100 | 5 | 99 |
| 7* | 50 | 120 | 4 | 100 | 5 | 99 |
| 7* | 50 | 120 | 4 | 40 | 5 | 100 |
| 7* | 50 | 120 | 4 | 60 | 5 | 100 |
| 8* | 50 | 120 | 4 | 100 | 5 | 99 |
| 8* | 50 | 120 | 4 | 40 | 5 | 99 |
| 8* | 50 | 120 | 4 | 60 | 5 | 100 |
| 9* | 50 | 120 | 4 | 100 | 5 | 99 |
| 10* | 50 | 120 | 4 | 100 | 5 | 98 |

*inventive compound Olefin: n-octene (Oxeno GmbH) Solvent: toluene Ligand/rhodium ratio (L/Rh): 5:1

As is evident from Table 1, a very good yield was achieved with all compounds according to the invention. In addition, a reduction of the rhodium concentration from 100 ppm to 60 ppm and 40 ppm is compensated for by the high activity of the ligands such that yields of greater than 90% were achieved.

This plays an important role, particularly in large-scale use, since it is preferable to use as small amounts as possible of the expensive rhodium metal and still obtain high yields of desired product.

TABLE 2

| Ligand | p (bar) | T (° C.) | t (h) | [Rh] (ppm) | L/Rh | Yield (%) |
|---|---|---|---|---|---|---|
| 3* | 20 | 120 | 4 | 100 | 5 | 86 |
| 4* | 20 | 120 | 4 | 100 | 5 | 91 |
| 7* | 20 | 120 | 4 | 100 | 5 | 98 |
| 8* | 20 | 120 | 4 | 100 | 5 | 98 |
| A | 20 | 120 | 4 | 100 | 5 | 29 |
| B | 20 | 120 | 4 | 100 | 5 | 21 |

*inventive compound Olefin: n-octene (Oxeno GmbH) Solvent: toluene Ligand/rhodium ratio (L/Rh): 5:1

Four of the compounds listed in Table 1 were also tested at a lower pressure (20 bar). All four compounds in this case also led to very good yields.

A considerably improved yield was achieved with the inventive compounds than with the comparative compounds (A) and (B).

It could be shown on the basis of the experiments described above, that the stated object is achieved by the compounds according to the invention.

European patent application EP14196197.9 filed Dec. 4, 2014, is incorporated herein by reference.

Numerous modifications and variations on the present invention are possible in light of the above teachings. It is therefore to be understood that within the scope of the appended claims, the invention may be practiced otherwise than as specifically described herein.

1. A compound having one of the structures I or II: 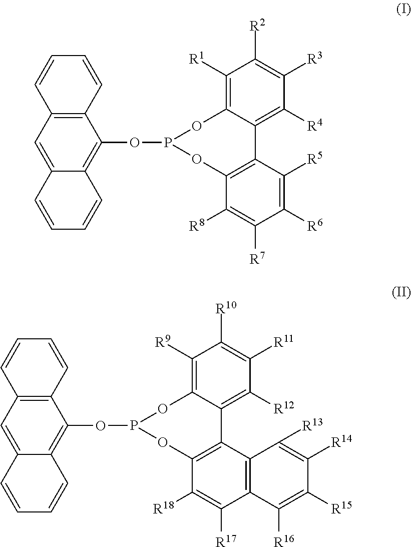
wherein
R¹, R², R³, R⁴, R⁵, R⁶, R⁷, R⁸ are each independently selected from the group consisting of:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —CN, —NH₂, and —N[(C₁-C₁₂)-alkyl]₂;
R⁹, R¹⁰, R¹¹, R¹², R¹³, R¹⁴, R¹⁵, R¹⁶, R¹⁷, R¹⁸ are each independently selected from the group consisting of:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —NH₂, and —N[(C₁-C₁₂)-alkyl]₂;
wherein the alkyl and aryl groups may be substituted.

2. The compound according to claim 1,
wherein R¹, R², R³, R⁴, R⁵, R⁶, R⁷, R⁸ are each independently selected from the group consisting of:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —CO—(C₁-C₁₂)-alkyl, —CO—(C₆-C₂₀)-aryl, and —N[(C₁-C₁₂)-alkyl]₂.

3. The compound according to claim 1,
wherein R⁹, R¹⁰, R¹¹, R¹², R¹³, R¹⁴, R¹⁵, R¹⁶, R¹⁷, R¹⁸ are each independently selected from the group consisting of:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —CO—(C₁-C₁₂)-alkyl, —CO—(C₆-C₂₀)-aryl, and —N[(C₁-C₁₂)-alkyl]₂.

4. The compound according to claim 1,
wherein R¹, R², R³, R⁴, R⁵, R⁶, R⁷, R⁸ are each independently selected from the group consisting of:
—H, —(C₁-C₁₂)-alkyl, —O—(C₁-C₁₂)-alkyl, —O—(C₆-C₂₀)-aryl, —(C₆-C₂₀)-aryl, —S-alkyl, -S-aryl, and halogen.

5. The compound according to claim 1,
wherein R⁹, R¹⁰, R¹¹, R¹², R¹³, R¹⁴, R¹⁵, R¹⁶, R¹⁷, R¹⁸ are each independently selected from the group consisting of:
—H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, and halogen.

6. The compound according to claim 1,
wherein R¹, R², R³, R⁴, R⁵, R⁶, R⁷, R⁸ are each independently selected from the group consisting of:
—H, —(C₁-C₁₂)-alkyl, —O—(C₁-C₁₂)-alkyl, and —O—(C₆-C₂₀)-aryl.

7. The compound according to claim 1,
wherein R⁹, R¹⁰, R¹¹, R¹², R¹³, R¹⁴, R¹⁵, R¹⁶, R¹⁷, R¹⁸ are each independently selected from the group consisting of:
—H, —(C₁-C₁₂)-alkyl, —O—(C₁-C₁₂)-alkyl, and —O—(C₆-C₂₀)-aryl.

8. The compound according to claim 1,
wherein R¹, R², R³, R⁴, R⁵, R⁶, R⁷, R⁸ are each independently selected from the group consisting of:
—H, —(C₁-C₁₂)-alkyl, and —O—(C₁-C₁₂)-alkyl.

9. The compound according to claim 1,
wherein R⁹, R¹⁰, R¹¹, R¹², R¹³, R¹⁴, R¹⁵, R¹⁶, R¹⁷, R¹⁸ are each independently selected from the group consisting of:
—H, —(C₁-C₁₂)-alkyl, and —O—(C₁-C₁₂)-alkyl.

10. The compound according to claim 1,
having a structure selected from the group consisting of the following structures 1 to 10:

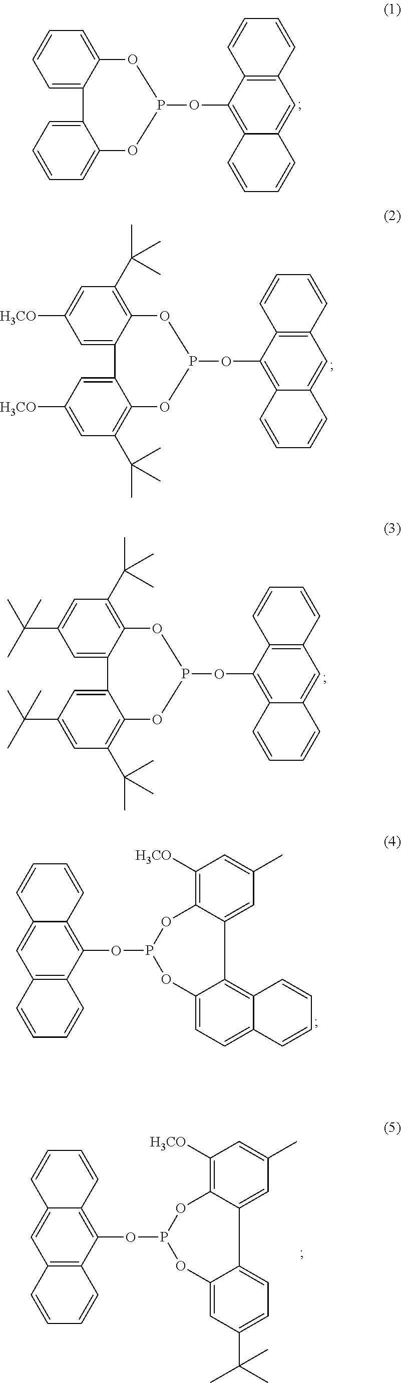

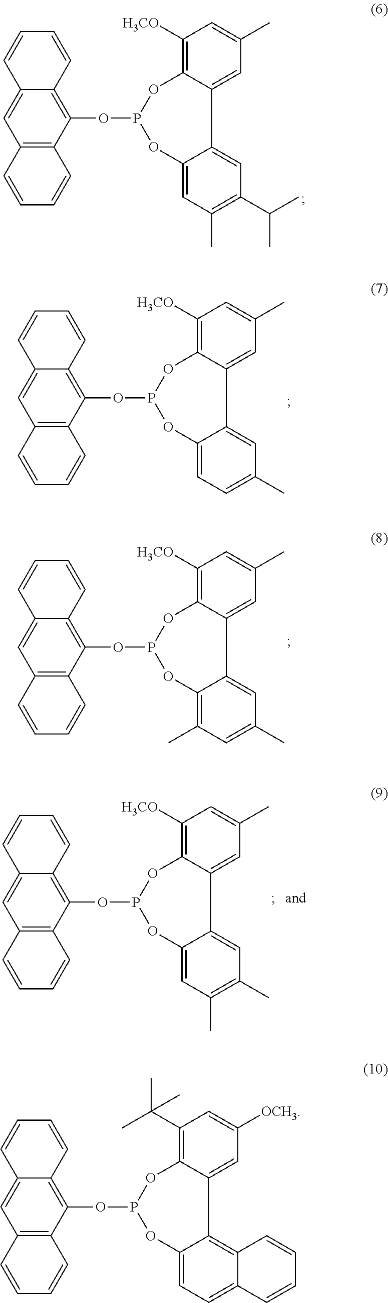 [SMILES: COc1cc(C)cc2c1op(Oc1c3ccccc3cc3ccccc13)oc1cc(C)c(C(C)C)cc12 ; COc1cc(C)cc2c1op(Oc1c3ccccc3cc3ccccc13)oc1ccc(C)cc12 ; COc1cc(C)cc2c1op(Oc1c3ccccc3cc3ccccc13)oc1c(C)cc(C)cc12 ; COc1cc(C)cc2c1op(Oc1c3ccccc3cc3ccccc13)oc1cc(C)c(C)cc12 ; COc1cc(C(C)(C)C)c2op(Oc3c4ccccc4cc4ccccc34)oc3ccc4ccccc4c3c2c1]

11. The compound according to claim 1,
wherein the compound has the structure I.

12. The compound according to claim 1,
wherein the compound has the structure II.

13. A complex, comprising:
a compound according to claim 1; and
a metal atom selected from the group consisting of: Rh, Ru, Co, and Ir.

14. The complex according to claim 13,
wherein said compound has the structure (I).

15. The complex according to claim 13,
wherein said compound has the structure (II).

16. A catalyst for catalyzing a hydroformylation reaction, comprising: the compound according to claim 1.

17. The catalyst according to claim 16,
wherein said compound has the structure (I).

18. The catalyst according to claim 16,
wherein said compound has the structure (II).

19. A process for hydroformylation of an olefin, comprising:
a) initially charging an olefin into a reactor;
b) adding
i) a complex according to claim 13;
or
ii) a compound according to claim 1 and a substance having a metal atom selected from the group consisting of: Rh, Ru, Co, and Ir;
c) feeding into said reactor H₂ and CO, to obtain a reaction mixture;
d) heating the reaction mixture, to obtain conversion of the olefin to an aldehyde.
